(19) United States
(12) Patent Application Publication
(10) Pub. No.: US 2011/0269226 A1
(43) Pub. Date: Nov. 3, 2011

(54) Microfluidic Continuous Flow Device for Culturing Biological Material

(75) Inventors: Danny Van Noort, Singapore (SG); Hanry Yu, Singapore (SG)

(73) Assignee: Agency for Science, Technology and Research, Connexis, Singapore (SG)

(21) Appl. No.: 13/061,236

(22) PCT Filed: Aug. 27, 2008
(86) PCT No.: PCT/SG2008/000318

§ 371 Date: Jul. 11, 2011

(51) Int. Cl. C12N 5/09; A01K 61/00; C12M 3/00

(52) U.S. Cl. 435/325; 435/294.1; 119/217

(57) ABSTRACT

The present invention refers to a microfluidic continuous flow devices for culturing biological material each comprising a cultivation chamber being dimensioned to retain a biological material and having an inlet and an outlet to allow flow of a cultivation medium through the cultivation chamber. The present invention also refers to a method using the microfluidic continuous flow device of the present invention and the uses for these devices. In one example a microfluidic continuous flow device of the present invention is connected to a gradient generator.

### FIELD OF THE INVENTION

The present invention refers to a microfluidic continuous flow device for culturing biological material each comprising a cultivation chamber being dimensioned to retain a biological material and having an inlet and an outlet to allow flow of a cultivation medium through the cultivation chamber. The present invention also refers to a method using the microfluidic continuous flow device of the present invention and to assays in which such a method and device is used. In one example a microfluidic continuous flow device of the present invention is connected to a gradient generator.

### BACKGROUND TO THE INVENTION

Technological advances led by achievements in microfabrication and tissue engineering have provided the tools needed to create microscale devices for conducting many types of laboratory assays. Microfluidic devices have been developed for conducting a variety analytical/biochemical laboratory processes on a very small scale. Sometimes called “lab-on-a-chip,” the microscale perfusion devices sometimes consist only of microscope slide/credit card-sized units containing compartments that are connected by channels through which fluid flow is maintained by a micropump. Known examples include microfluidic devices for conducting immunoassays, PCR sample preparation, DNA separation, or identifying protein-protein interactions.

Use of microfluidic flow devices in research and industry allow to reduce sample and liquid volumes due to miniaturization of the device and the liquid guiding structures. Smaller sample sizes and miniaturized devices also allow for carrying out of more parallel examinations at the same time which again helps to reduce the overall costs.

Cell-based microfluidic devices, the application of microfluidic technology to cell culture-based assays, are also described as “cell chips,” “cell biochips,” or “microbioreactors.” These microscale cell assay devices can be practical tools for the rapid screening of chemicals and drugs.

The present invention provides microfluidic devices for cultivation of different biological materials.

### SUMMARY OF THE INVENTION

In a first aspect the present invention refers to a microfluidic continuous flow device for culturing biological material, comprising:
a concentration gradient generator having at least two outlets;
at least two cultivation chambers being dimensioned to retain a biological material in each of the cultivation chambers;
   wherein each of the at least two cultivation chambers has a circumferential wall, wherein the circumferential wall has at least one inlet and at least one outlet in order to allow flow of a cultivation medium through each of the at least two cultivation chambers;
   wherein each inlet of said at least two cultivation chambers is fluidly connected to a different outlet of said at least two outlets of said concentration gradient generator.

In another aspect the present invention refers to a microfluidic continuous flow device for culturing biological material, comprising:
a cultivation chamber being dimensioned to retain biological material in the cultivation chamber;
   wherein the cultivation chamber has a circumferential wall, wherein the circumferential wall has at least one inlet and at least one outlet in order to allow flow of a cultivation medium through the cultivation chamber;
   biological material which is retained in the cultivation chamber;
   wherein the biological material is selected from the group of a tumor spheroid and an organism in an embryonic stage.

In a further aspect the present invention refers to a method of culturing biological material in a microfluidic continuous flow device, comprising:
providing the microfluidic continuous flow device comprising:
   at least two cultivation chambers being dimensioned to retain a biological material in each of the cultivation chambers;
      wherein each of the at least two cultivation chambers has a circumferential wall, wherein the circumferential wall has at least one inlet and at least one outlet in order to allow flow of a cultivation medium through each of the at least two cultivation chambers;
   a concentration gradient generator having at least two outlets;
      wherein each outlet of the concentration gradient generator is fluidly connected to a different inlet of one of the at least two cultivation chambers; and
   a biological material which is retained in each of the cultivation chambers;
introducing a cultivation medium and a chemical substance into the concentration gradient generator whereby at the at least two outlets of the concentration gradient generator a mixture of the cultivation medium and the chemical substance is obtained, wherein each mixture comprises the chemical substance in a different concentration;
letting each of the mixtures flow through a different of the cultivation chambers which retain the biological material.

In still another aspect the present invention refers to a method of culturing biological material in a microfluidic continuous flow device, comprising:
providing the microfluidic continuous flow device comprising:
   a cultivation chamber being dimensioned to retain biological material in the cultivation chamber;
      wherein the cultivation chamber has a circumferential wall, wherein the circumferential wall has an inlet and an outlet in order to allow flow of cultivation medium through the cultivation chamber;
providing a biological material which is retained in the cultivation chamber,
   wherein the biological material is selected from the group of a tumor spheroid and an organism in an embryonic stage;
letting a mixture of a cultivation medium and a chemical substance flowing through the cultivation chamber which retains the biological material.

In still another aspect the present invention refers to a kit comprising a microfluidic continuous flow device of the present invention and in still another aspect the present invention refers to the use of a microfluidic continuous flow device of the present invention for biological assays. Such assays can, for example, be high throughput drug screening assays, assays for wastewater analysis or assays testing the biological effect of at least one chemical substance. In the last mentioned assay the chemical substance may be a pharmaceutical composition, a compound which is or which is suspected to be necessary for the cultivation of the biological material and which is initially not comprised in the cultivation medium; a compound which is or which is suspected to be necessary for the metabolism of the biological material and which is initially not comprised in the cultivation medium; a compound or composition which is or which is suspected to be teratogenic, cancerogenic, mutagenic, psychogenic, toxic; and mixtures thereof.

### BRIEF DESCRIPTION OF THE DRAWINGS

The invention will be better understood with reference to the detailed description when considered in conjunction with the non-limiting examples and the accompanying drawings, in which:

FIG. 1 shows a cross-sectional view of a microfluidic continuous flow device. The cultivation chamber 18 comprises a fish embryo 24. The inlet 15 and the outlet 27 (dotted lines) are connected to an inlet 14 and outlet 28 channel forming an integral part of the microfluidic continuous flow device. Those channels 14 and 28 are connected to further fluid connectors forming the outside part 13 and 23 of the inlet and outlet channel, respectively. The outside tubing 12 and 22 are short metal tubes connected to a Tygon™ tubing 10 and 20. Inlet 15 and outlet 27 are oriented to allow flow of medium diagonal across the well to ensure that the medium envelopes the embryo. The microfluidic continuous flow device 30 comprises a cover or upper layer 16 forming the upper closure of the cultivation chamber 18 and the bottom layer 26 forming the bottom closure of the cultivation chamber 18.

FIG. 2 shows a top view of a microfluidic continuous flow device comprising an area 130 including multiple cultivation chambers 18 and their respective inlet 13 and outlet channels 23 and an area 140 and 110 comprising the concentration gradient generator 140 and the inlets 110 of the concentration gradient generator. The entire length of this device indicated by the arrow at the right side of FIG. 2 is about 40 mm long and about 20 mm wide which means that such a device can fit on a standard microscope slide having a dimension of 25×76 mm.

FIG. 3 shows a top view of the concentration gradient generator 140 shown in FIG. 2. The concentration gradient generator illustrated in FIG. 3 produces a sigmodial concentration distribution of a chemical substance introduced through one of the two inlets I and J of the concentration gradient generator. A cultivation medium for the biological material in the cultivation chamber is introduced through the other inlet of the concentration gradient generator. The cultivation medium does not comprise the chemical substance introduced through the other inlet of the concentration gradient generator. The concentration distribution of the chemical substance at the outlets of the concentration gradient generator C1 to C8 is 1=c₀ (C1), 63/64 (C2), 57/64 (C3), 42/64 (C4), 32/64 (C5), 7/64 (C6), 1/64 (C7) and 0 (no chemical substance=pure cultivation medium) (C8).

FIG. 4 is a graphical illustration of the distribution pattern of a chemical substance fed into the concentration gradient generator referred to in FIG. 3. The y-axis shows the relative fluorescence units (RFU) vs. the channel outlet number C1 to C8. The blank squares show the theoretical calculated values for the expected concentration at the separate outlets while the filled black diamonds indicate the values for the concentration of a chemical substance measured at the separate outlets C1 to C8. In this example the chemical substance and the cultivation medium were represented by a green and red food dye introduced into the concentration gradient generator.

FIG. 5 shows three cultivation chambers 18. Each cultivation chamber has eight inlets and eight outlets. Every inlet or outlet is connected to an inlet and outlet channel, respectively. Two inlet or outlet channels merge into a merged inlet or outlet channel forming a bifurcated inlet or outlet channel unit. The encircled area in FIG. 5 shows a bifurcated outlet channel unit 230 comprising of two outlet channels 231 and 232 merging into a merged outlet channel 233. Each merged channel of an inlet or outlet channel unit merges again with another channel of a neighboring inlet or outlet channel unit to form a further bifurcated inlet or outlet channel unit. Merging of inlet or outlet channels continuous until only one merged inlet or outlet channel is left as illustrated in FIG. 5. This network of inlet or outlet channels 210, 220 connected to the eight inlets of the cultivation chamber serves to distribute the medium in the cultivation chamber more evenly. The different gray colors illustrate the different polymer layers of the microfluidic continuous flow device which have been manufactured separately and assembled together after manufacturing.

FIG. 6 and FIG. 7 show the flow profile of a liquid medium within the cultivation chamber in grayscales. Brighter areas indicate a higher velocity of the medium in the cultivation chamber. In FIG. 6 inlet 313 and outlet 323 are located at the bottom of the cultivation chamber at opposite sides while in FIG. 7 inlet 313 and outlet 323 are located at opposing lateral sides at a different height, i.e. in this case at opposite corners of the circumferential wall of the cultivation chamber.

FIG. 8 shows different orientations of inlet (black diamond on the left side of the cultivation chamber 18) and outlet (black diamond on the right side of the cultivation chamber 18) in the side wall of a cultivation chamber 18. The direction of flow of the medium is indicated by arrows.

FIG. 9 shows a diagram illustrating the results of an experiment in which a medeka fish embryo was subjected to different concentrations of TAA (triamcinolone acetonide, 0-3%) over different times (0 to 25 hours) in a cultivation chamber illustrated in FIG. 1. TAA affects the liver by inducing lipid peroxidation. After 21 hours the medeka embryos subjected to TAA at a concentration of 3 v/v % died while after 25 hours the medeka embryos subjected to TAA at a concentration of 1.5, 2 and 2.5 v/v % also died.

FIG. 10 shows a diagram illustrating the results of an experiment in which a medeka fish embryo was subjected in a cultivation chamber illustrated in FIG. 1 to different concentrations of ethanol (EtOH) (0 to 5 v/v %) over a time of 25 hours. Similar to TAA, ethanol also damages the liver. The higher the concentration of the EtOH, the intoxicated the embryo is, while at the highest concentration (4.27% and 5%) the organs seem to be affected as well.

FIG. 11 shows a microfluidic continuous flow device having multiple cultivation chambers which form a first row of cultivation chambers. The cultivation chambers of the first row are connected via their outlets to the cultivation chambers of a second row which again is connected to the cultivation chambers of a third row. The microfluidic continuous flow device shown in FIG. 11 comprises 8 rows with cultivation chambers. The number of cultivation chambers in one row is variable. Each of the cultivation chambers shown in FIG. 11 comprises more than one inlet and outlet, namely eight inlets and eight outlets. Those in and outlets are connected to in and outlet channels which merge to form bifurcated in or outlet channel units. Merging of in and outlet channels continuous until only one inlet channel or outlet channel is left. The resulting single inlet or outlet channel is fluidly connected to the previous or next inlet or outlet channel of a cultivation chamber. The inlet channels of the first row of cultivation chambers can be connected to a concentration gradient generator as shown in FIG. 11.

FIG. 12A shows the setup of a linear gradient generator. The substance to be diluted into different concentrations and fed into the concentration gradient generator exits such a linear concentration gradient generator in concentrations of 0, 0.25, 0.5, 0.75 and 1 relative to the initial concentration. FIG. 12B shows the setup of a logarithmic gradient generator as referred to in the article of Pihl, J., Sinclair, J. et al. (2005, Anal. Chem., vol. 77, p. 3897). This exemplary logarithmic concentration gradient generator consists of six input wells which can clearly be seen in FIG. 12B and which are connected through a microfluidic dilution network to an open volume. The lower part of FIG. 12B shows a magnified section of one of the dilution elements. Eight mixing channels are entering the dilution generation from above, are joined, and split up into nine channels that exit at the bottom of the generation. The microfluidic network consists of 18 generations of dilution and mixing, and at the exit into the open volume, the network has expanded to 24 channels. In this exemplary device mixing takes place by diffusion. This logarithmic concentration gradient generator is made of PDMS and has a dynamic range of nearly 5 orders of magnitude from one single concentration. The channel dimensions are 45×61 μm (w×h) except for the more complex elements such as the dilution generations and the turns within the mixing channels.

### DETAILED DESCRIPTION OF THE INVENTION

In a first aspect the present invention refers to a microfluidic continuous flow device for culturing biological material, comprising:
a concentration gradient generator having at least two outlets;
at least two cultivation chambers being dimensioned to retain a biological material in each of the cultivation chambers;
   wherein each of the at least two cultivation chambers has a circumferential wall, wherein the circumferential wall has at least one inlet and at least one outlet in order to allow flow of a cultivation medium through each of the at least two cultivation chambers;
   wherein each inlet of the at least two cultivation chambers is fluidly connected to a different outlet of the at least two outlets of the concentration gradient generator.

Such a microfluidic based platform allows constant perfusion, small size, disposability, parallel analysis and low consumption of cultivation medium. The “continuous flow” of cultivation medium through the cultivation chamber also allows not only a continuous and fresh supply of substances such as, e.g., nutrients and oxygen which are needed for the cultivation and development of the biological medium but also the possibility to adjust the conditions in the cultivation chamber very quickly, for example to change concentrations of certain ingredients in the cultivation medium or to add further substances, such as chemical substances mentioned further below.

Due to the use of the concentration gradient generator the above device allows cultivation of biological material under varying conditions. Concentration gradient generators are known in the art (e.g. U.S. Pat. No. 7,314,070; Lin, F., Saadi, W., et al., 2004, Lab on a Chip, vol. 4, p. 164; Walker, G. M., Sai, J., et al., 2005, Lab on a Chip, vol. 5, p. 611) and comprise in general at least two inlets for supply of two liquid streams. The liquid stream introduced into a concentration gradient generator at the first inlet differs from the liquid stream introduced into the concentration gradient generator at the second inlet insofar that at least one substance (also called herein test substance or chemical substance) is comprised only in the liquid stream introduced into the concentration gradient generator through the second inlet. This results in different mixtures of those two liquid streams exiting the concentration gradient generator at the outlets of the concentration gradient generator wherein the at least one substance is comprised in every or at least some mixtures exiting through the outlet in a different concentration depending on the mixture of both liquid streams within the concentration gradient generator.

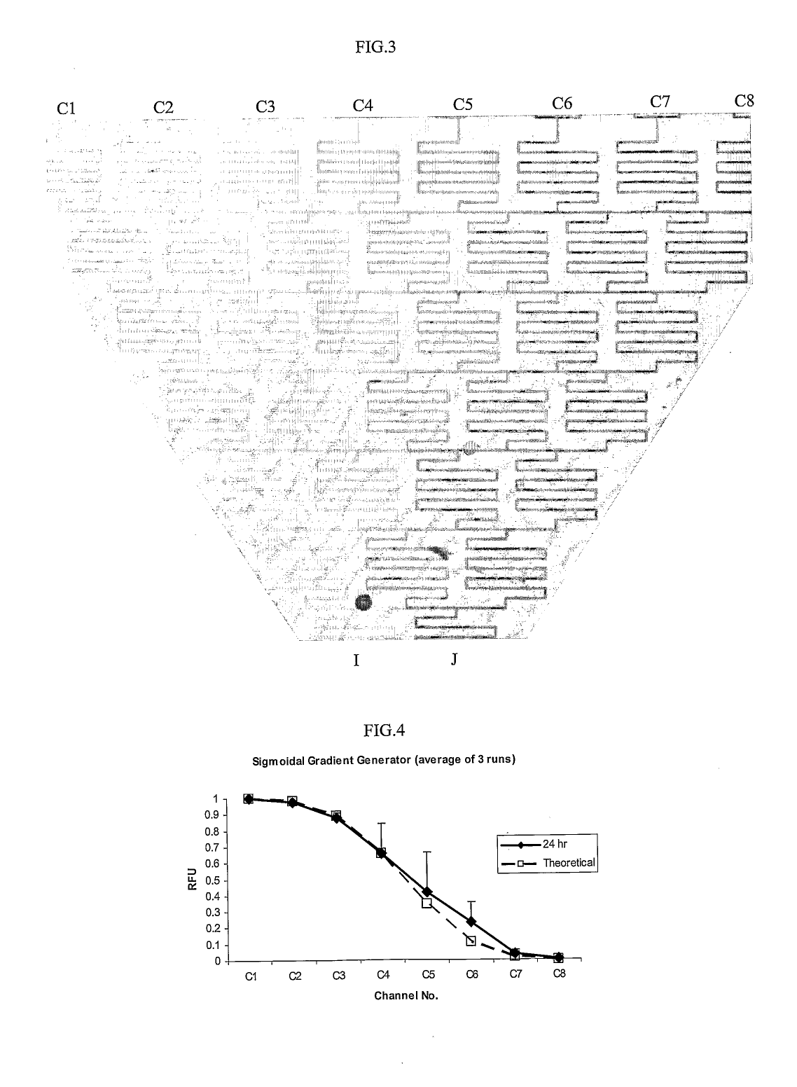

An exemplary concentration gradient generator is illustrated in FIG. 3. Two liquid streams are introduced via the inlets I and J into the concentration gradient generator shown in FIG. 3, wherein one liquid stream contains a substance A at a concentration c₀ and the other liquid stream does not contain this substance. By repeatedly dividing and mixing the two input liquid streams in different proportions of the channel system of the concentration gradient generator an arbitrary number of liquid streams can be created with varying concentrations of substance A at the outlets of the concentration gradient generator C1 to C8. The concentration gradient generator illustrated in FIG. 3 for example provides a sigmoidal distribution of substance A at the outlets C1 to C8 (y=A2+(A1−-A2)/(1+(x/x₀)̂p). The concentration distribution of substance A at the outlets of the concentration gradient generator C1 to C8 is 1=c₀ (C1), 63/64 (C2), 57/64 (C3), 42/64 (C4), 32/64 (C5), 7/64 (C6), 1/64 (C7) and 0 (no substance A=pure cultivation medium) (C8). FIG. 4 compares the calculated concentrations which are expected to be measured at the outlets C1 to C8 with the measured concentrations at the outlets C1 to C8.

Besides a sigmoidal concentration distribution it is also possible that the gradient generator provides any other concentration distribution, such as a linear, an exponential, a logarithmic, a quadratic, a sinusoidal, a squared or a cubed distribution. It is for example also possible to use a flow rate gradient generator. As different biological material reacts differently to shear forces, an additional function can be implemented into the microfluidic continuous flow device of the present invention. Thus, the present invention also refers to a microfluidic continuous flow device comprising a flow rate gradient generator or a concentration and flow rate gradient generator. An example for a flow rate gradient generator is referred to in the article of Kim, L., Vahey, M. D., et al. (2006, Lab on a Chip, vol 6, p. 394).

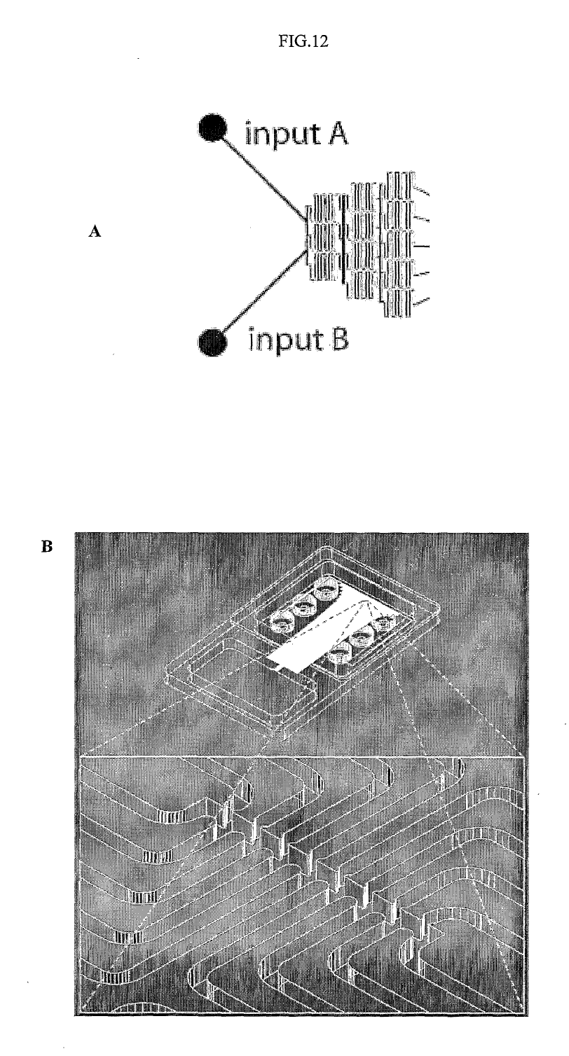

An example for a logarithmic concentration gradient generator is described for example in the article of Pihl, J., Sinclair, J. et al. (2005, Anal. Chem., vol. 77, p. 3897). The design of this logarithmic concentration gradient generator is shown in FIG. 12B. Briefly, this exemplary logarithmic concentration gradient generator consists of six inputs connected through a microfluidic dilution network to an open volume. The microfluidic network consists of 18 generations of dilution and mixing, and at the exit into the open volume, the network has expanded to 24 channels. In this exemplary device mixing takes place by diffusion.

In another example a linear gradient generator can be used as for example described by Walker, G. M., Sai, J., et al. (2005, supra). In brief, the two input streams entering the concentration gradient generator are divided and mixed in a device illustrated in FIG. 12A until five different mixtures of the solutions entering the concentration gradient generator through input A and input B are obtained. One of the solutions entering the concentration gradient generator comprises the substance whose concentration is supposed to be varied. The concentrations of this substance at the five outputs of the linear concentration gradient generator illustrated in FIG. 12A are 0, 0.25, 0.5, 0.75 and 1.

The channels of the concentration gradient generator have a variable width. In one example the width is less than about 1 mm while in another example the width of the channels is less than about 100 μm.

It is also possible to provide a microfluidic flow device comprising not only one concentration gradient generator but 2, 3, 4, 5, 6 or even more. This provides for example the option to generate different concentration gradients within one microfluidic flow device, i.e. linear, logarithmic etc. It is also possible that all concentration gradient generators provide the same concentration gradient. In this case some of the cultivation chambers connected to these concentration gradient generators are supplied with liquid streams having all the same concentration of a certain substance or certain substances.

It is further possible that the concentration gradient generator comprises more than two inlets in order to vary the concentration of several test substances at the same time. However, varying the concentration of different substances at the same time can also be achieved by introducing a mixture of different test substances into the concentration gradient generator through one of the at least two inlets.

In another case it might be desirable to change the concentration of a test substance or a mixture of test substances in a cultivation chamber during the experiment. In such a case the outlet of a cultivation chamber is disconnected from the outlet of the concentration gradient generator and re-connected to another outlet of the same or a different concentration gradient generator providing the same test substance or mixture of test substances in another concentration. It is also possible that this other outlet provides a solution comprising a different test substance or mixture of test substances.

The at least two outlets of the concentration gradient generator are fluidly connected to a cultivation chamber which retains the biological material. In one aspect, the present invention is directed to a microfluidic continuous flow device comprising a concentration gradient generator which comprises multiple outlets and wherein the microfluidic continuous flow device comprises multiple cultivation chambers wherein each of the inlets of the multiple cultivation chambers is fluidly connected to a different outlet of the concentration gradient generator.

In this configuration it is possible to supply several cultivation chambers retaining the same or different biological material with a liquid stream of cultivation medium comprising a certain substance, such as substance A, or mixture of substances at a different concentration.

In another aspect the present invention refers to a microfluidic continuous flow device for culturing biological material, comprising:
a cultivation chamber being dimensioned to retain biological material in the cultivation chamber;
   wherein the cultivation chamber has a circumferential wall, wherein the circumferential wall has at least one inlet and at least one outlet in order to allow flow of a cultivation medium through the cultivation chamber;
   biological material which is retained in the cultivation chamber;
   wherein the biological material is selected from the group of a tumor spheroid and an organism in an embryonic stage.

In this aspect of the present invention a cultivation chamber retaining biological material is provided without the use of a concentration gradient generator. In another example this microfluidic continuous flow device comprises multiple cultivation chambers wherein each cultivation chamber has a circumferential wall and wherein each of the circumferential walls has at least one inlet and at least one outlet. Each inlet can be connected to the same cultivation medium source or container which has the effect that the same cultivation medium including any substance comprised therein flows through all cultivation chambers. In another example each inlet of the cultivation chambers is connected to a different medium source or container which has the effect that each cultivation chamber is perfused with a different cultivation medium or a cultivation medium comprising at least one substance, which is not comprised in the cultivation medium or is comprised in a different concentration, which does not flow through another cultivation chamber. When multiple cultivation chambers are comprised in the microfluidic continuous flow device it is also possible to fluidly connect each inlet of each cultivation chamber with a different outlet of a concentration gradient generator.

As mentioned above each outlet of the concentration gradient generator provides a liquid stream having, e.g., a substance A at a certain concentration. The connection between an outlet of the concentration gradient generator and the inlet of a cultivation chamber can be a channel having the same structure and dimensions as the channels of the concentration gradient generator. It is also possible that the width of a channel which is fluidly connecting an outlet of the concentration gradient generator and an inlet of a cultivation chamber has a different width. Increasing the width of the connecting channel relative to the width of the channel of the concentration gradient generator reduces the speed of the liquid inside the channel. Decreasing the width of the connecting channel relative to the width of the channel of the concentration gradient generator increases the speed of the liquid inside the channel.

In another example, an outlet of the concentration gradient generator splits up into several outlet channels which are all feeding the same liquid stream into a different cultivation chamber, i.e. one outlet of a concentration gradient generator is fluidly connected with more than one cultivation chamber, namely with at least two, three, four, five, six, seven, eight or even more.

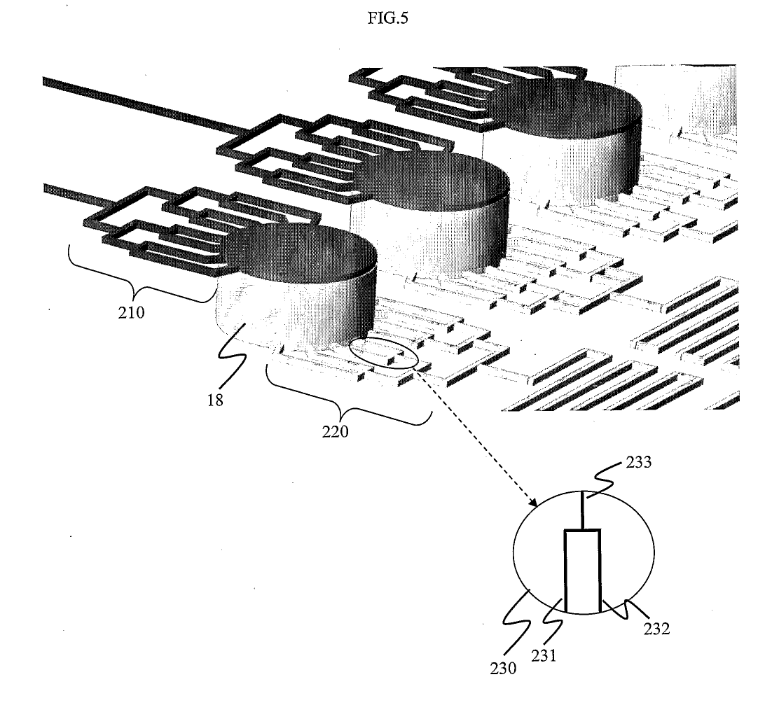

In another example, illustrated for example in FIG. 5, a cultivation chamber 18 comprises several inlets to ensure a good distribution of the liquid stream in the cultivation chamber. Thus, in one aspect the present invention refers to a microfluidic continuous flow device wherein each of the cultivation chambers comprises at least two inlets which are each connected to an inlet channel, wherein each inlet channel merges with the respective other inlet channel into a single merged inlet channel to form a bifurcated inlet channel unit. This single merged inlet channel is fluidly connected to an outlet of a concentration gradient generator. In another example, at least one or each of the cultivation chambers of the microfluidic continuous flow devices comprises multiple inlets which are each connected to an inlet channel, wherein each two of the multiple inlet channels form a bifurcated inlet channel unit and wherein each single merged inlet channel of a bifurcated inlet channel unit merges with a neighboring single merged inlet channel to form a further bifurcated inlet channel unit until only one single merged inlet channel unit remains. Such a construct can lead to a network of inlet channels and inlets of a cultivation chamber as shown in FIG. 5 (210).

A similar structure is also possible for the outlets of the cultivation chamber. Thus, in another example each of the cultivation chambers of the microfluidic continuous flow device comprises at least two outlets which are each connected to an outlet channel, wherein each outlet channel merges with the respective other outlet channel into a single merged outlet channel to form a bifurcated outlet channel unit. In still another example each of the cultivation chambers of the microfluidic continuous flow device comprises multiple outlets which are each connected to an outlet channel, wherein each two of the multiple outlet channels form a bifurcated outlet channel unit and wherein each single merged outlet channel of a bifurcated outlet channel unit merges with a neighboring single merged outlet channel to form a further bifurcated outlet channel unit until only one single merged outlet channel unit remains. Such a construct results in a network of outlet channels and outlets of a cultivation chamber as shown in FIG. 5 (220). An example of a bifurcated outlet channel unit 230 is illustrated in the circled area of FIG. 5. FIG. 5 shows a first 231 and second 232 outlet channel which merges into a merged outlet channel 233. The same construct of a bifurcated channel unit can also be found at the inlet side of the cultivation chamber.

Thus, it is possible that a cultivation chamber can comprise for example 2, 4, 6, 8 or even more inlets and outlets, respectively, which merge in the above described manner until only one inlet or outlet channel is left. It is also possible that the number of inlet and outlets of the cultivation chamber is different from each other.

As can be seen in FIG. 5, the multiple inlets and outlets are all located in one plane. However, instead of having several inlets and outlets in one plane it is also possible that the multiple inlets and/or outlets are distributed over the vertical axis of a cultivation chamber. In this case the cultivation medium can enter and exit the cultivation chamber at several points along the vertical axis of the cultivation chamber.

The inlet and outlet of at least one or of each of the cultivation chambers can be located at different positions in the circumferential wall of each of the cultivation chambers. The inlet can, for example, be located at the top of a cultivation chamber while the outlet is located at a lateral position of a cultivation chamber. In another example, the inlet and the outlet of the cultivation chamber are located at opposing sides in the circumferential wall of the cultivation chambers. That means for example that the inlet is located at the top while the outlet is located at the bottom of a cultivation chamber or that the inlet is located at the side of a cultivation chamber while the outlet is located at the opposite side.

In still another aspect the inlet and outlet of at least one or of each of the cultivation chambers are located at opposing lateral sites in a different height (when seen in a cross-section) in the circumferential wall of each of the cultivation chambers. Examples for the different positions of an inlet and an outlet are illustrated, for example, in FIG. 8. It should be noted that FIG. 8 shows only some possible examples and that the position of inlet and outlet is not limited to the exemplified positions given in FIG. 8 but inlet and outlet can be shifted “off center”, too.

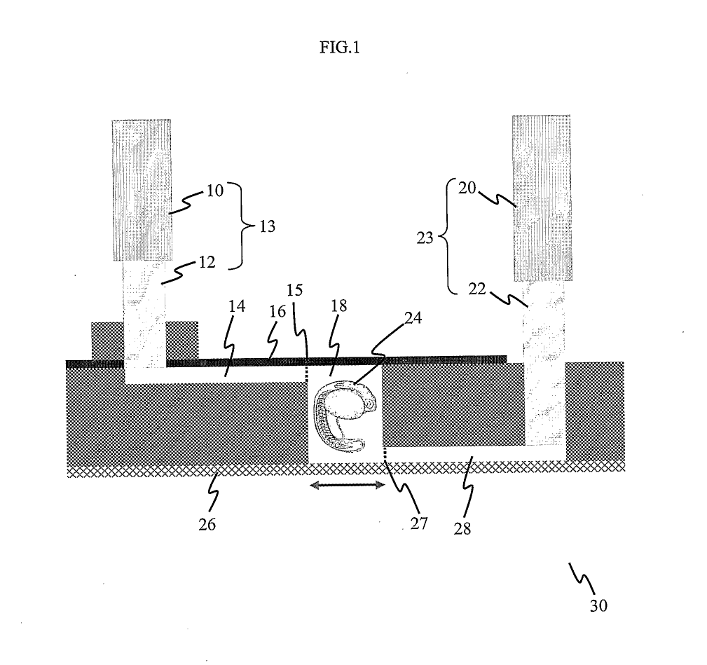

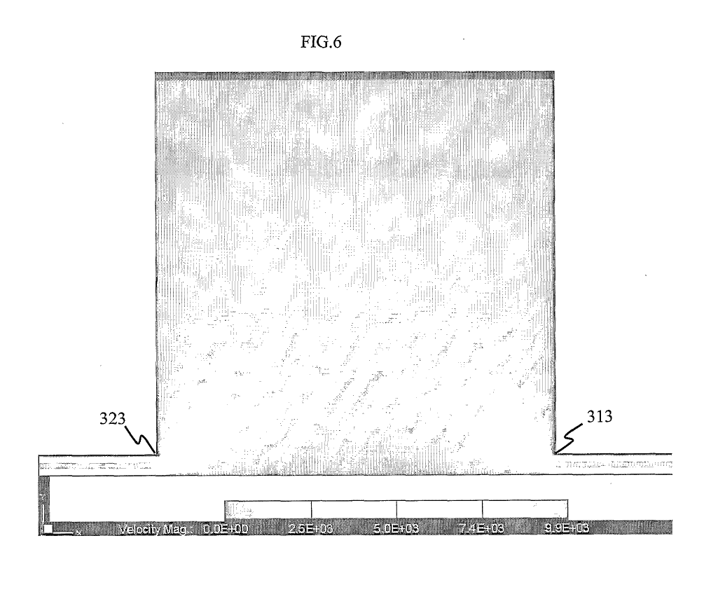

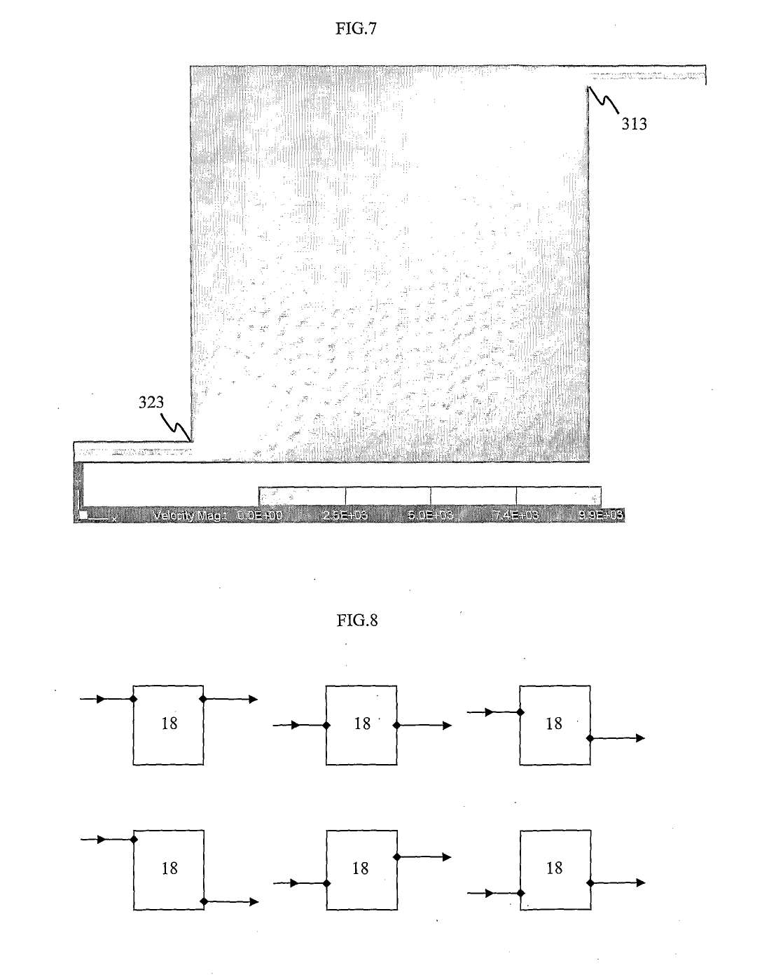

Positioning the inlet and outlet at different heights causes a different flow profile of the medium through the cultivation chamber as illustrated in FIGS. 6 and 7. In FIG. 6 the inlet 313 and the outlet 323 are located at the same height at the bottom of the sides of the cultivation chamber while in FIG. 7 the inlet 313 is located at the top right side of the cultivation chamber and the outlet 323 is located at the bottom left side of the cultivation chamber. As can be seen from the varying color profile in FIGS. 6 and 7, positioning the inlet and outlet at opposite lateral sites at different heights leads to a more evenly distribution of medium within the cultivation chamber. In case a biological material is located in the cultivation chamber as illustrated for example in FIG. 1 this means that the biological material is supplied with incoming medium at all sides, i.e. the liquid flow envelopes the biological material. Providing a continuous flow in the system ensures that transport of medium and other substances does not rely solely on diffusion but is actively driven. However, depending on the size and the shape of the biological material other positions of the inlet and outlet can also be possible.

Thus in one aspect the present invention refers to a microfluidic continuous flow device wherein the inlet and the outlet of at least one or of each of the cultivation chambers are located at different heights at substantially opposing sites of the circumferential wall of each of the cultivation chambers (in case of a chamber with a polygonal shape (base) such as a rectangular base, the inlet and the outlet can be arranged in opposing lateral sections of the wall. In case of a cylindrical cross-section/shape of a cultivation chamber, the inlet and outlet may be arranged substantial facing each other at opposing location in the circumferential wall. The height difference is adapted to allow an essentially diagonal flow of a cultivation medium through the cultivation chamber. Adapting the position of the at least one inlet and outlet in order to achieve a diagonal flow of the cultivation medium through the cultivation chamber has at the same time the effect that the amount of any substance comprised in the cultivation medium is (more) evenly distributed over the entire cultivation chamber.

It is also possible to position multiple inlets at different positions and heights of the cultivation chamber in order to achieve an even more thorough distribution of the medium in the cultivation chamber.

The cultivation chamber can in general be of any shape as long as it is dimensioned to retain a biological material in the cultivation chamber. The cultivation chamber should be dimensioned in order to retain the biological material in a position that allows for example optical analysis of the biological material retained in the cultivation chamber. The shape (seen in cross section) of the cultivation chamber can be for example polygonal or a trapezoid. In another example, the shape (seen in cross section) of the cultivation chamber can be a semi-circular, or circular cross section. Cultivation chambers of other polygonal cross-sections, such as a triangular, square, rectangular, pentagonal, hexagonal, octagonal, oblong, ellipsoidal etc. are also possible.

The size of the cultivation chamber can be varied depending on the desired need and purpose and the size of the biological material cultured therein. In general, the cultivation chamber is dimensioned in order to retain the biological material located in the cultivation chamber. The size of the cultivation chamber should not only allow to locate the biological material in the chamber but also to retain it in position so as to allow optical analysis of the biological material located in it. At the same time the cultivation chamber should allow expansion of the biological material retained in the chamber. Therefore, the cultivation chamber can have for example a diameter or width (depending on the shape) which is between about 0.1 mm to about 10 mm. In one example the chamber is round and has a diameter of about 1.2 mm. The height of the chamber can be in the same range. In one example the cultivation chamber is about 2 mm high.

The substrate for manufacturing the microfluidic continuous flow device inclusive the cultivation chambers and the concentration gradient generator may be molded using any type of material which can be made into a microfluidic continuous flow device of the invention. In one example the material is chosen to allow observation of cells. Such materials include polymers, glass, silicone or certain types of metal. Therefore, in one aspect the present invention refers to a microfluidic continuous flow device wherein at least on side or defined section of one side of each of the circumferential walls of the cultivation chambers is transparent or translucent. In one example, the bottom or top side is transparent or translucent. For example, in the example illustrated in FIG. 1 the top side 16 can be transparent or translucent and/or the bottom side 26 can be transparent or translucent.

In one embodiment, the material for forming the substrate is a biocompatible material. A biocompatible material includes, but is not limited to, glass, silicon and a polymerisable material. The polymerisable material includes, but is not limited to, monomers or oligomeric building blocks (i.e. every suitable precursor molecule) of polycarbonate, polyacrylic, polyoxymethylene, polyamide, polybutyl enterephthalate, polyphenylenether, polydimethylsiloxane (PDMS), mylar, polyurethane, polyvinylidene fluoride (PVDF), flourosilicone or combinations and mixtures thereof. In some examples, the biocompatible material comprises PVDF and/or PDMS. Advantages of PVDF and PDMS are their cheap price and superior biocompatibility. Furthermore, as they are transparent, they conveniently allow direct morphological observation of the biological material under an observation device, e.g. a microscope, to be carried out. In one example the microfluidic continuous flow device is made of poly(-dimethylsiloxane) (PDMS).

Furthermore, the microfluidic continuous flow device can comprise a cover and/or bottom layer (see e.g. FIGS. 1, 16 and 26) forming the top and/or bottom of the cultivation chamber. The cover layer can have any suitable optical transparency. A fully opaque cover or one which is transparent, or one which is translucent material (thereby permitting the transmission of a certain amount of light), may all be used. In a further example, the top and/or bottom layer may comprise a biocompatible material that is transparent or at least substantially translucent in order that the device is compatible for use with optical microscopes which can provide a backlight that can be directed through the cultivation chamber in order to provide a bright view of the processes occurring in the cultivation chamber during its use.

Another aspect of the invention concerns the fabrication of the above described microfluidic continuous flow devices. The template for creating the device of the invention can be fabricated according to any technique known in the art, such as photolithography, etching, electron-beam lithography, laser ablation, hot embossing, etc. depending on the material used. For example, when fabricating devices using Si templates in microscale and nanoscale, it is possible to use laser ablation, etching or hot embossing, and electron-beam lithography respectively. Templates can also be manufactured using epoxy based negative resists with high functionality, high optical transparency and sensitivity to near UV radiation, such as photoresists of the SU-8 series from MicroChem Corp. (Newton, Mass., US). The above techniques are known in the area of microelectronics and microfabrication. After creating the template the microfluidic continuous flow device is then created by replica molding of, for example, poly(-dimethylsiloxane) (PDMS) on the template. In one example, the silicon templates can for example be fabricated by standard deep reactive ion etching (DRIE) process.

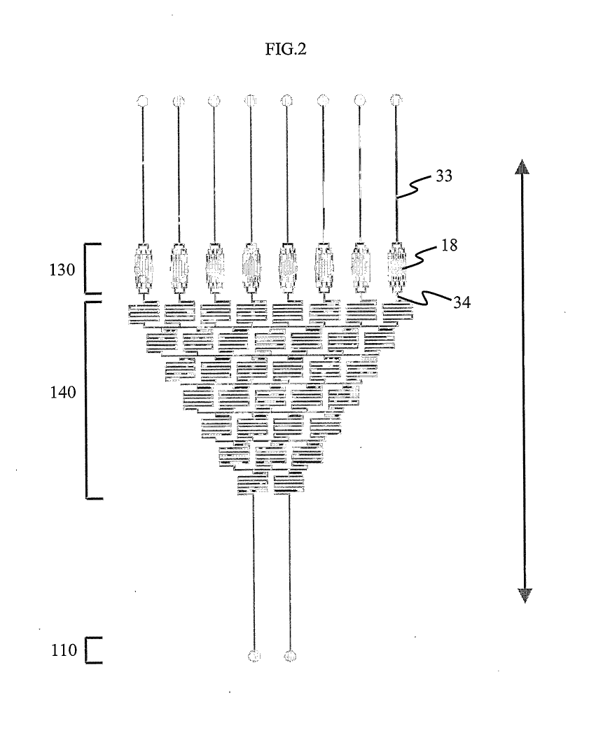

In order to simulate physiological flow conditions, the delivery of cultivation medium and control of cultivation medium flow in the present device can be achieved in any technique known in the art. One method is to adjust the height of the fluid medium reservoir which is fluidly connected to the microfluidic continuous flow device of the present invention. This would correspondingly adjust the hydrostatic pressure, and thus the flow rate of the fluid medium in the device. Alternatively, the flow rate can be adjusted by use of an actuating device e.g. a pump. One or more pumps may be incorporated into the device according to any known microfabrication technique. Examples of pumps which may be used include micromachined pumps, syringe pumps, diaphragm pumps, reciprocating pumps and other pumping means known to those skilled in the art. It is also possible to induce the flow of cultivation medium through a channel via capillary action. Cultivation medium flow in the device can be kept laminar in order to avoid any turbulence. In one example the flow of cultivation medium through the channel is driven by syringe pumps which are used to withdraw the cultivation medium out of the outlet channels of the microfluidic continuous flow device (23 in FIG. 2), which means that those pumps create a negative pressure in the channel system which drives the cultivation medium flow.

The biological material which is retained in the cultivation chamber includes, but is not limited to tumor spheroids and an organism in an embryonic stage. The organism in an embryonic stage includes, but is not limited to amphibian eggs, fish eggs, insect eggs and mammalian eggs. Examples for fish eggs include, but are not limited to an egg of a zebrafish (Danio rerio), an egg of a medaka (Oryzias latipes), an egg of a giant danio (Devario aequipinnatus), and an egg of a fish from the family Tetraodontidae (puffer fish). An example for an amphibian egg can include, but is not limited to toad eggs, frog eggs, an egg of Caenorhabditis elegans (C. elegans) and salamander eggs. Examples for an insect egg include, but are not limited to an egg from a fruit fly (Drosphila melanogaster). In some examples the organism can be a mammalian embryo except embryos of humans. It is also possible to use Caenorhabditis elegans (C elegans) for cultivation in the cultivation chamber of the microfluidic continuous flow device of the present invention. C. elegans is about 1 mm long and is used as model organism for studying cell differentiation.

The above fish species zebrafish, medaka, giant danio and embryos from the family Tetraodentidae are suitable vertebrate model organisms with similar organ systems and gene sequences to humans. The embryos of these fishes are optically transparent enabling organ visualisation. For example, zebrafish and medaka fish have embryonic development similar to the one of a human embryo and are therefore suitable for substitution of human models to study developmental defects caused, for example by drug candidates.

This platform allows for the creation of specific microenvironments in the cultivation chambers in which embryos reside. These fish embryos can be treated with small molecules and drugs for example for high-throughput analysis and for the identification and validation of drugs. High-throughput methodologies for use in these organisms include, but are not limited to, phenotype-based visualization, transcript studies using low-density DNA microarrays or proteomic analysis. The embryonic development, ex utero, of for example, medaka and zebrafish is 9 to 11 and 2 days, respectively, making those organisms very suitable for the cultivation in the cultivation chamber of the microfluidic continuous flow device of the present invention. Due to their small egg size (about 700 μm to about 1000 μm) they are also particularly suitable for analysis in a microfluidic continuous flow device of the present invention. To follow the development of these fishes it is possible to use transgenic animals. For example, by using a reporter protein (e.g., green fluorescence protein GFP) it is possible to follow the development effect of certain drugs on these organisms in the cultivation chamber.

With the device of the present invention it is also possible to further develop mature fish in the cultivation chamber and study development differences. For this purpose the fish egg is retained in the cultivation chamber until it hatches. The hatched young fishes can then be taken out of the cultivation chamber for further cultivation or for physiological or anatomical examination. Deformations that have been induced during cultivation of the embryos in the cultivation chamber might become visible only in a phase of the fish development. Accordingly, in one aspect of the present invention, one or both sides of the cultivation chamber which are not connected to an inlet or an outlet is adapted to be opened and closed.

The ex vivo development of transparent fish embryos in the cultivation chamber of the microfluidic continuous flow device allows the direct and dynamic observation of cellular processes in normal and perturbed states. Further examples of using fish embryos like the one referred to above for ex vivo analysis are provided by Love, D. R., Pichler, F. B., et al. (2004, Current Opinion in Biotechnology, vol. 15, p. 564), Langheinrich, U. (2003, BioEssays, vol. 25, no. 9, p. 904), Oxendine, S. L., Cowden, J., et al. (2006, NeuroToxicology, vol. 27, p. 840), Teuschler, L. K., Gennings, C., et al. (2005, Chemosphere, vol. 58, p. 1283), Lin, C. C., Michelle, N. Y., et al. (2007, Toxicology and Applied Pharmacology, vol. 222, p. 159), and Joakim Larsson, D. G., Fredriksson, S., et al. (2006, Environmental Toxicology and Pharmacology, vol. 22, p. 338).

Tumor spheroids are aggregates made up of tumour cells, or cell lines. The tumor spheroids can be selected from every kind of cancer tumor. Such a cancer can include, but is not limited to a basal cell carcinoma, bladder cancer, bone cancer, brain cancer, CNS cancer, breast cancer, cervical cancer, colon cancer, rectum cancer, connective tissue. cancer, esophageal cancer, eye cancer, kidney cancer, larynx cancer, liver cancer, lung cancer, Hodgkin's lymphoma, non-Hodgkin's lymphoma, melanoma, myeloma, leukemia, oral cavity cancer, ovarian cancer, pancreatic cancer, prostate cancer, rhabdomyosarcoma, skin cancer, stomach cancer, testicular cancer, neoplasia or uterine cancer.

In case the microfluidic continuous flow device comprises a plurality of cultivation chambers (that means at least two cultivation chambers), each of the cultivation chambers can comprise the same or different biological material. In another example only some of the multiple cultivation chambers comprises the same while other cultivation chambers comprise different biological material.

FIG. 1 shows a cross sectional view through an illustrative embodiment of a microfluidic continuous flow device 30 of the present invention comprising a cultivation chamber retaining a biological material 24 which is in this case a fish embryo. The device 30 comprises a cultivation chamber 18 comprising an inlet 15 at the upper left side of the cultivation chamber 18 which is connected to the inlet channels 13 and 14. Reference sign 13 represents the part of an inlet channel located outside the microfluidic continuous flow device 30 which is made in this example of PDMS. The part of the inlet channel located outside the device 13 consists in this example of a Tygon™ tubing 10 which is connected to a short metal tube 12 connecting the Tygon™ tubing 10 with the inlet channel 14 located inside the microfluidic continuous flow device 30. The inlet channel 14 is fluidly connected to the inlet 15 of the cultivation chamber 18. The outlet 27 of the cultivation chamber is located at the bottom right side of the cultivation chamber 18 and is connected with the outlet channel 28 located inside the microfluidic continuous flow device 30. The inlet channel 28 is connected with the outlet channel 23 which comprises the metal tube 22 and the Tygon™ tubing 20. Inlet 15 and outlet 27 of the cultivation chamber are oriented in order to allow a diagonal flow pattern through the cultivation chamber which ensures that a mixture of cultivation medium and chemical substance envelopes the embryo retained in the cultivation chamber 18. The microfluidic continuous flow device 30 further comprises a cover layer 16 and a bottom layer 26 which can both be made of a transparent material. The bottom layer 26 and the cover layer 16 close up the cultivation chamber 18 at the bottom and the top, respectively. The width of the cultivation chamber in this example is about 1.2 mm which wide enough to allow retaining of a 1 mm fish embryo 24 in the cultivation chamber 18.

FIG. 2 shows a top view of a microfluidic continuous flow device comprising an area 130 including multiple cultivation chambers 18 and their respective inlet channel network 34 and outlet channel network 33 and an area 140 and 110 comprising the concentration gradient generator 140 and the inlets 110 of the concentration gradient generator. All parts of the device are integrally molded in one plane. Other than the microfluidic continuous flow device shown in FIG. 1, inlet and outlet channels of the cultivation chamber are located inside the microfluidic continuous flow device. During operation of this device a chemical substance A is introduced into the concentration gradient generator through a first inlet at a concentration c₀ while the cultivation medium is introduced into concentration gradient generator through a second inlet. After reaching the eight different outlets of the concentration gradient generator different liquid streams of cultivation medium comprising eight different concentrations of substance A are obtained and fed into the cultivation chambers 18 through the network of inlet channels connected with the different inlet networks 34 of the eight cultivation chambers. This system can be easily scaled up by providing either several microfluidic flow devices of this kind or by increasing the number of addressable cultivation chambers in the microfluidic continuous flow device.

In another aspect, the present invention refers to a method of culturing biological material in a microfluidic continuous flow device, comprising:
providing the microfluidic continuous flow device comprising:
   at least two cultivation chambers being dimensioned to retain a biological material in each of the cultivation chambers;
      wherein each of said at least two cultivation chambers has a circumferential wall, wherein said circumferential wall has at least one inlet and at least one outlet in order to allow flow of a cultivation medium through each of the at least two cultivation chambers;
   a concentration gradient generator having at least two outlets;
      wherein each outlet of the concentration gradient generator is fluidly connected to a different inlet of one of the at least two cultivation chambers; and
   a biological material retained in each of said cultivation chambers;
introducing a cultivation medium and a chemical substance into the concentration gradient generator whereby at the at least two outlets of the concentration gradient generator a mixture of the cultivation medium and the chemical substance is obtained, wherein each mixture comprises the chemical substance in a different concentration;
letting each of the mixtures flow through a different of the cultivation chambers which retain the biological material.

In still another aspect the present invention refers to a method of culturing biological material in a microfluidic continuous flow device, comprising:
providing the microfluidic continuous flow device comprising:
   a cultivation chamber being dimensioned to retain biological material in the cultivation chamber;
      wherein the cultivation chamber has a circumferential wall, wherein the circumferential wall has an inlet and an outlet in order to allow flow of cultivation medium through the cultivation chamber;
   a biological material which is retained in the cultivation chamber,
      wherein the biological material is selected from the group of a tumor spheroid and an organism in an embryonic stage;
letting a mixture of a cultivation medium and a chemical substance flow through the cultivation chamber which retains the biological material.

The chemical substance can be any molecule which has or is suspected to have an effect on the biological material retained in the cultivation chamber. Such a chemical substance can include, but is not limited to a pharmaceutical composition, a compound which is or which is suspected to be necessary for the cultivation of the biological material and which is initially not comprised in the cultivation medium; a compound which is or which is suspected to be necessary for the metabolism of the biological material and which is initially not comprised in the cultivation medium; a compound or composition which is or which is suspected to be teratogenic, cancerogenic, mutagenic, psychogenic or toxic, or mixtures thereof. Such a chemical substance can also be a gaseous substance.

The above list of chemical substances shows that the microfluidic continuous flow device of the present invention is intended to be used for screening of all kind of substances who can have an effect on a biological material, such as one of the aforementioned organisms. The microfluidic continuous flow device is thus designed to replace in vivo tests partly or completely. It is especially suitable for parallel screening of large amounts of compounds, for example from existing compound libraries which can comprise up to 7 million different compounds. Screening the reaction of more complex organism instead of testing the reaction of single cells to a chemical substance can provide data which are easier transferable to the human system.

For example, Lin, C. C., Michelle, N. Y., et al. (2007, supra) tested the effect of carbaryl (acetylcholinesterase inhibitor) on the early development of zebrafish. LC50 and EC50 values for carbaryl have been determined. Red blood cell accumulation and delayed hatching are only some of the effects which have been observed when using zebrafish as organism for testing of the effect of carbaryl. Teuschler, L. K., Gennings, C., et al. (2005, supra) used the developing medeka for studying the effect of benzene and toluene. The experiments were designed to obtain further data on the toxicity of those substances for the developing fish embryo.

The above described microfluidic continuous flow devices can be used for any biological assays such as, but not limited to, high throughput drug screening assays, wastewater and drinking water analysis assays, assays testing of the biological effect of at least one chemical substance. To name only a few examples, this at least one chemical substance may be a pharmaceutical compound or composition, a compound which is or which is suspected to be necessary for the cultivation of the biological material and which is initially not comprised in the cultivation medium; a compound which is or which is suspected to be necessary for the metabolism of the biological material and which is initially not comprised in the cultivation medium; a compound or composition which is or which is suspected to be teratogenic, cancerogenic, mutagenic, psychogenic, toxic; or mixtures thereof.

The microfluidic continuous flow device can be used for real time imaging of the biological material retained in the cultivation chamber of the microfluidic continuous flow device. It may, for example, be used to obtain high-resolution images and videos of the embryos or parts of the embryo, such as the liver, heart, or screening neurotoxic effects.

The system can also be used for dose-dependent toxicity studies on the biological material retained in the cultivation chamber. It is also possible to target specific organs, such as liver, heart etc., by using transgenic organisms such as fish. As mentioned before, it is for example possible to use a reporter protein (e.g. green fluorescence protein (GFP) or yellow fluorescence protein (YFP)) which allows to follow the developmental effect of pharmaceutically active substances and drugs on the fish embryo. It is also possible to let the embryos further develop to mature fish and investigate developmental differences (developmental biology).

In the microfluidic continuous flow device of the present invention drugs can be administered in different concentrations to the biological material, such as embryos. While some drugs might not have an immediate affect at the embryonic stage, they can have an affect at a more mature stage of the development. For example, with a reversible bonding technique of the glass slide covering the wells on the chip, it is possible to retrieve drug-treated embryos and let them mature to the adult stage. At this point, drug-related developmental defects can be probed on the adult fish.

In another aspect the present invention refers to a kit comprising a microfluidic flow device of the present invention. The kit can further comprise a biological material and suitable cultivation medium for culturing of the biological material.

By “consisting of” is meant including, and limited to, whatever follows the phrase “consisting of”. Thus, the phrase “consisting of” indicates that the listed elements are required or mandatory, and that no other elements may be present.

By “comprising” it is meant including, but not limited to, whatever follows the word “comprising”. Thus, use of the term “comprising” indicates that the listed elements are required or mandatory, but that other elements are optional and may or may not be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

At first manufacturing of a microfluidic continuous flow device is illustrated on the basis of the microfluidic continuous flow device shown in FIG. 2.

The microfluidic continuous flow device was made from of PDMS (Polydimethylsiloxane, Sylgard 184, Dow-Corning, Mich,, USA). The device consists of 3 parts: the concentration gradient generator 140, the area including the cultivation chambers 130 and the outlet channels 23. Those parts are divided over three layers. The first layer comprises the concentration gradient generator and the top layer of the cultivation chamber, the second layer comprises the main body of the cultivation chamber and the third layer comprises the bottom layer of the cultivation chamber and the output channels (see for example FIG. 5). For all layers a mixture of 1:10 (1 curing agent, e.g. Sylgard 184 (Corning, USA): 10 parts PDMS) was degassed and spin-coated on a template at 100 rpm for 2 minutes, resulting in a 500 μm thick layer. This was cured for 30 min at 80° C. A thin sheet of PDMS was used to cover the outlet layer. Holes where punched for the 2 inputs to the gradient generator and the 8 outlet openings with a punch (Technical Innovations Inc., Brazoria, Tex., US). Hereafter, the cultivation chambers were punched using a 1.2 mm diameter punch. The PDMS structure was then irreversibly bonded to a microscope slide by treating both with a Corona Surface Treater (Electro-Technic Products, Inc., Chicago. Ill. USA).

The microfluidic continuous flow device as shown in FIG. 1 has been manufactured in the same way. The tubings (Tygon™ tubing) were connected via small metal tubes (from New England Small Tube Company, USA), which fit exactly in the punched holes (made by a punch from Innovative Technologies, USA).

Introduction of a Fish Embryo Into a Cultivation Chamber

A medeka fish embryo was introduced into the cultivation chamber using a pipette. The fish embryos maintain the same size until they hatch. Once all the embryos are transferred to the 8 wells of the microfluidic continuous flow device shown in FIG. 1, a coverslip is placed over the wells to seal them off.

Visualization of the Fish Embryo in the Cultivation Chamber

It was demonstrated that from the cultivation chamber of the microfluidic continuous flow device shown in FIG. 1 images and videos can be obtained. A video of the heart beat (130 beats/min) of the Medaka embryo with a 150× stereo microscope (Zeiss) was made. By using a high-speed camera, variations in the heart rate due to drug administration. insecticides or environmental changes can be easily monitored (data not shown).

Furthermore, a z-stack was made with a confocal microscope (Zeiss) of a life transgenic Medaka embryo at 200×. In this manner a 3D model of the fluorescence liver can be constructed. Any change in the liver size or shape due to drugs administered can be monitored over time (data not shown).

Effect of Chemical Substances on Fish Embryo Development

In two different experiments EtOH (ethanol, 0-5%) and TAA (triamcinolone acetonide, 0-3%) were used as chemical substances to test the reaction of the fish embryo retained in the cultivation chamber of the microfluidic continuous flow device shown in FIG. 1. Both substances affect the liver (TAA induces lipid peroxidation which leads to liver damage). An over-dosage of both will lead to death. The flow rate in the microfluidic continuous flow device is about 1 ml/2.5 hours or 400 μl/h.

The higher the concentration of the EtOH, the intoxicated the embryo is, while at the highest concentration (4.27% and 5%) the organs seem to be affected as well.

The zebra fish embryos are obtained from the zebra fish facility of the Institute of Molecular and Cell Biology (IMCB) in Singapore. They were induced to breeding by over feeding them. In general, the embryos can be harvest at different stadiums, depending on the development one wants to see.

When observing the effects of TAA, after 21 hours, at stage 38, 3% concentration (the highest concentration) caused the death of the Medaka embryo. After 25 hours, the Medaka embryos in the 4 highest concentrations had died (FIG. 9).

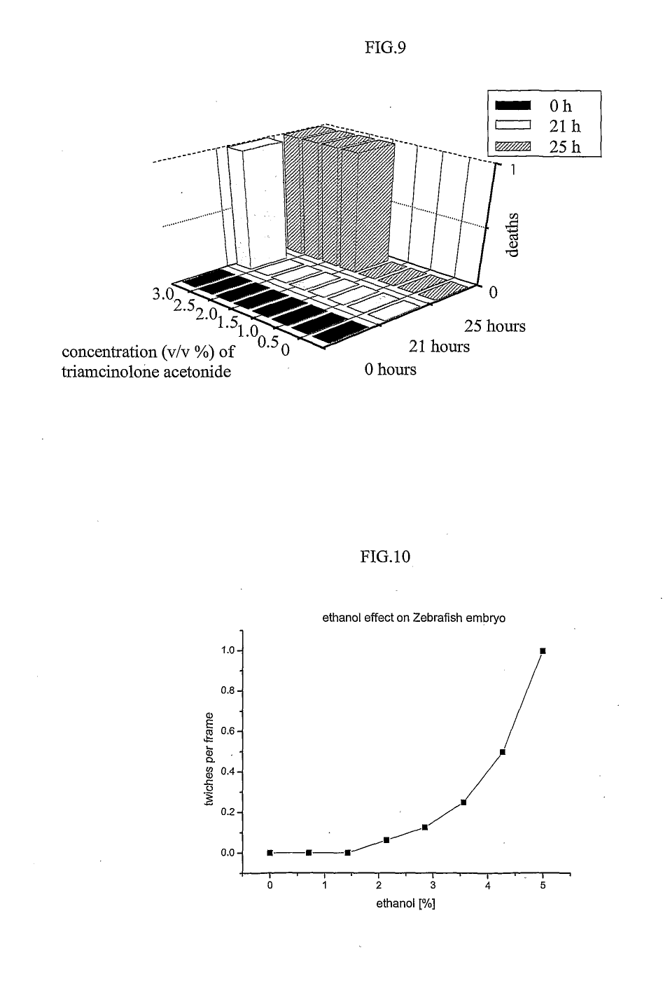

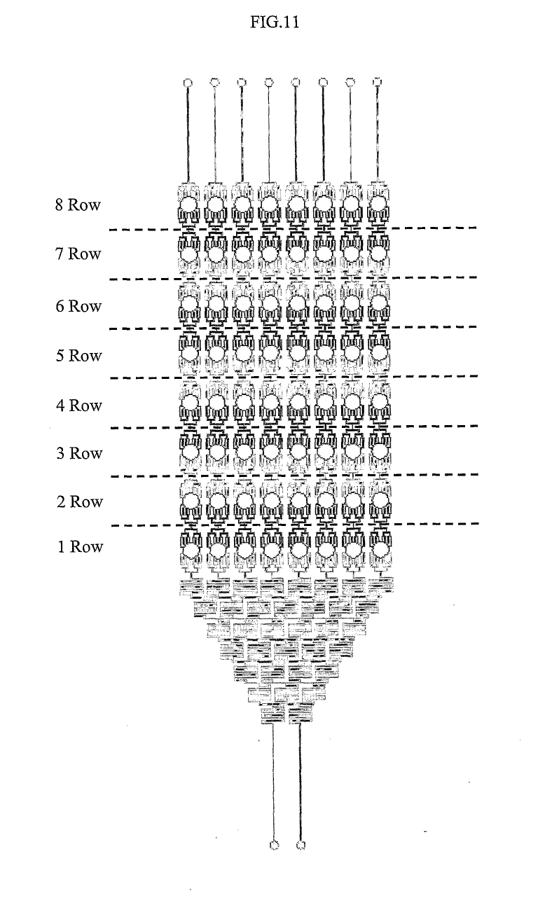

As can be seen from FIG. 10, the higher the ethanol concentration, the more the embryo twitches (moves) in the chamber. Thus the number of twitches rose with the ethanol concentration. Furthermore the organs turned black in the higher concentrations.

1. A microfluidic continuous flow device for culturing biological material, comprising:
a concentration gradient generator having at least two outlets;
at least two cultivation chambers being dimensioned to retain a biological material in each of said cultivation chambers;
wherein each of said at least two cultivation chambers has a circumferential wall, wherein said circumferential wall has an inlet and an outlet which are located at different heights of said circumferential wall of said cultivation chamber so as to allow a diagonal flow of a cultivation medium through said cultivation chamber;
wherein each inlet of said at least two cultivation chambers is fluidly connected to a different outlet of said at least two outlets of said concentration gradient generator.

2. The microfluidic continuous flow device according to claim 1, wherein each of said at least two cultivation chambers retains a biological material selected from the group consisting of a tumor spheroid and an organism in an embryonic stage.

3. The microfluidic continuous flow device according to claim 2, wherein said organism in an embryonic stage is selected from the group consisting of an amphibian egg, fish egg, insect egg and a mammalian egg.

4. The microfluidic continuous flow device according to claim 3, wherein said fish is selected from the group consisting of a zebrafish (Danio rerio), a medaka (Oryzias latipes), a giant danio (Devario aequipinnatus), and a fish from the family Tetraodontidae.

5. The microfluidic continuous flow device according to claim 1, wherein said concentration gradient generator comprises multiple outlets and wherein said microfluidic continuous flow device comprises multiple cultivation chambers wherein each of said inlet of said multiple cultivation chambers is fluidly connected to a different outlet of said concentration gradient generator.

6. The microfluidic continuous flow device according to claim 1, wherein said cultivation chambers which are fluidly connected to said concentration gradient generator form a first row of cultivation chambers and wherein said device further comprises a second row of cultivation chambers, wherein the number of said cultivation chambers of said second row of cultivation chambers equals the number of cultivation chambers in said first row of cultivation chambers, wherein each inlet of each of said cultivation chambers of said second row of cultivation chambers is fluidly connected to said respective outlet of said previous cultivation chamber in said first row of cultivation chambers.

7. The microfluidic continuous flow device according to claim 6, wherein said device comprises multiple rows of cultivation chambers.

8. A microfluidic continuous flow device for culturing biological material, comprising:
a cultivation chamber being dimensioned to retain biological material in said cultivation chamber;
wherein said cultivation chamber has a circumferential wall, wherein said circumferential wall has an inlet and an outlet which are located at different heights of said circumferential wall of said cultivation chamber so as to allow a diagonal flow of a cultivation medium through said cultivation chamber;
biological material which is retained in said cultivation chamber;
wherein said biological material is selected from the group of a tumor spheroid and an organism in an embryonic stage.

9. The microfluidic continuous flow device according to claim 8, wherein said organism in an embryonic stage is selected from the group consisting of an amphibian egg, fish egg, insect egg and a mammalian egg.

10. The microfluidic continuous flow device according to claim 9, wherein said fish is selected from the group consisting of a zebrafish (Danio rerio), a medaka (Oryzias latipes), a giant danio (Devario aequipinnatus), and a fish from the family Tetraodontidae.

11. The microfluidic continuous flow device according to claim 8, wherein said microfluidic continuous flow device comprises multiple cultivation chambers having a circumferential wall and wherein each of said circumferential walls has an inlet and an outlet.

12. The microfluidic continuous flow device according to claim 11, wherein each inlet of each of said cultivation chambers is fluidly connected to the same cultivation medium source.

13. The microfluidic continuous flow device according to claim 11, wherein each inlet of each of said cultivation chambers is fluidly connected to a different cultivation medium source.

14. The microfluidic continuous flow device according to claim 11, wherein each inlet of each of said cultivation chambers is fluidly connected to a different outlet of a concentration gradient generator.

15. The microfluidic continuous flow device according to claim 12, wherein said multiple cultivation chambers form a first row of cultivation chambers, and wherein said device comprises a second row of cultivation chambers, wherein the number of cultivation chambers in said second row equals the number of cultivation chambers in said first row and wherein each inlet of said cultivation chambers in said second row is fluidly connected with said outlet of each of said respective cultivation chambers in said first row.

16. The microfluidic continuous flow device according to claim 15, wherein said device comprises multiple rows of cultivation chambers.

17. The microfluidic continuous flow device according to claim 1, wherein said inlet and said outlet of at least one of said cultivation chambers is located at different positions in said circumferential wall of each of said chambers.

18. The microfluidic continuous flow device according to claim 17, wherein said inlet and said outlet of said at least one cultivation chamber is located at opposing sites in said circumferential wall of each of said chambers.

19. The microfluidic continuous flow device according to claim 1, wherein each of said cultivation chambers has a polygonal shape seen in cross-section.

20. The microfluidic continuous flow device according to claim 1, wherein each of said cultivation chambers has a rectangular shape or a trapezoidal shape or a pentagonal shape or a hexagonal shape or an octagonal shape or an oblong shape or an ellipsoidal shape.

21. The microfluidic continuous flow device according to claim 1, wherein each of said cultivation chambers comprises at least two inlets which are each connected to an inlet channel, wherein each inlet channel merges with said respective other inlet channel into a single merged inlet channel to form a bifurcated inlet channel unit.

22. The microfluidic continuous flow device according to claim 21, wherein each of said cultivation chambers comprises multiple inlets which are each connected to an inlet channel, wherein each two of said multiple inlet channels form said bifurcated inlet channel unit and wherein each single merged inlet channel of said bifurcated inlet channel unit merges with a neighboring single merged inlet channel to form a further bifurcated inlet channel unit until only one single merged inlet channel unit remains.

23. The microfluidic continuous flow device according to claim 1, wherein each of said cultivation chambers comprises at least two outlets which are each connected to an outlet channel, wherein each outlet channel merges with said respective other outlet channel into a single merged outlet channel to form a bifurcated outlet channel unit.

24. The microfluidic continuous flow device according to claim 23, wherein each of said cultivation chambers comprises multiple outlets which are each connected to an outlet channel, wherein each two of said multiple outlet channels form said bifurcated outlet channel unit and wherein each single merged outlet channel of said bifurcated outlet channel unit merges with a neighboring single merged outlet channel to form a further bifurcated outlet channel unit until only one single merged outlet channel unit remains.

25. The microfluidic continuous flow device according to claim 1, wherein said cultivation chambers comprise the same or different biological material.

26. The microfluidic continuous flow device according to claim 1, wherein at least one side or a defined section of one side of each of said circumferential walls of said cultivation chambers is transparent or translucent.

27. The microfluidic continuous flow device according to claim 26, wherein the bottom or top are transparent or translucent.

28. The microfluidic continuous flow device according to claim 1, wherein one or both of the sides of the cultivation chamber which are not connected to said inlet(s) or said outlet(s) is/are adapted to be opened and closed.

29. A method of culturing biological material in a microfluidic continuous flow device, comprising:
providing said microfluidic continuous flow device comprising:
at least two cultivation chambers being dimensioned to retain a biological material in each of said cultivation chambers;
wherein each of said at least two cultivation chambers has a circumferential wall, wherein said circumferential wall has an inlet and an outlet which are located at different heights of said circumferential wall of said cultivation chamber so as to allow a diagonal flow of a cultivation medium through said cultivation chamber;
a concentration gradient generator having at least two outlets;
wherein each outlet of said concentration gradient generator is fluidly connected to a different inlet of one of said at least two cultivation chambers; and
a biological material retained in each of said cultivation chambers;
introducing a cultivation medium and a chemical substance into said concentration gradient generator whereby at said at least two outlets of said concentration gradient generator a mixture of said cultivation medium and said chemical substance is obtained, wherein each mixture comprises said chemical substance in a different concentration;
letting each of said mixtures flow through a different of said cultivation chambers which retain said biological material.

30. A method of culturing biological material in a microfluidic continuous flow device, comprising:
providing said microfluidic continuous flow device comprising:
a cultivation chamber being dimensioned to retain biological material in said cultivation chamber;
wherein said cultivation chamber has a circumferential wall, wherein said circumferential wall has an inlet and an outlet which are located at different heights of said circumferential wall of said cultivation chamber so as to allow a diagonal flow of a cultivation medium through said cultivation chamber;
a biological material which is retained in said cultivation chamber,
wherein said biological material is selected from the group of a tumor spheroid and an organism in an embryonic stage;
letting a mixture of a cultivation medium and a chemical substance flow through said cultivation chamber which retains said biological material.

31. The method according to claim 30, wherein said organism in an embryonic stage is selected from the group consisting of an amphibian egg, fish egg, insect egg and a mammalian egg.

32. The method according to claim 31, wherein said fish is selected from the group consisting of a zebrafish (Danio rerio), a medaka (Oryzias latipes), a giant danio (Devario aequipinnatus), and a fish from the family Tetraodontidae.

33. The method according to claim 29, wherein said chemical substance is selected from the group consisting of a pharmaceutical composition, a compound which is or which is suspected to be necessary for the cultivation of said biological material and which is initially not comprised in said cultivation medium; a compound which is or which is suspected to be necessary for the metabolism of said biological material and which is initially not comprised in said cultivation medium; a compound or composition which is or which is suspected to be teratogenic, cancerogenic, mutagenic, psychogenic or toxic, and mixtures thereof.

34. The method according to claim 30, wherein said microfluidic continuous flow device comprises multiple cultivation chambers.

35. The method according to claim 29, wherein said chambers comprise the same or different biological material.

36. The method according to claim 19, wherein at least one side or a defined section of one side of each of said circumferential walls of said cultivation chambers is transparent or translucent.

37. A kit comprising:
a microfluidic flow device according to claim 1.

38. The kit according to claim 37, further comprising a biological material selected from the group consisting of a tumor spheroid and an organism in an embryonic stage.

39. The kit according to claim 37, further comprising a cultivation medium suitable for cultivation of said biological material.

40. A kit comprising:
a microfluidic flow device according to claim 8.

41. The kit according to claim 40, further comprising a cultivation medium suitable for cultivation of said biological material.

42. The kit according to claim 40, further comprising a concentration gradient generator having at least two outlets.

43. (canceled)

44. (canceled)
